(19) United States
(12) Patent Application Publication
(10) Pub. No.: US 2022/0028509 A1
(43) Pub. Date: Jan. 27, 2022

(54) SYSTEM AND METHOD FOR MATCHING MEDICAL CONCEPTS IN RADIOLOGICAL REPORTS

(71) Applicant: ALGOTEC SYSTEMS LTD., Ra'anana (IL)

(72) Inventor: Or ALMER, RA'ANANA (IL)

(21) Appl. No.: 17/296,688

(22) PCT Filed: Nov. 22, 2019
(86) PCT No.: PCT/EP2019/082288

§ 371 Date: May 25, 2021

Related U.S. Application Data
(60) Provisional application No. 62/771,308, filed on Nov. 26, 2018.

(51) Int. Cl. G16H 15/00; G16H 50/70

(52) U.S. Cl. CPC G16H 15/00; G16H 50/70

(57) ABSTRACT

A method of determining which concepts in a set of medical concepts pertain to an input text, comprising: a) creating a set of queries for each concept, each query being a string of two of the words in the concept; b) for each query, determining whether or not the input text includes all the words of that query, and calculating a sub-score indicating a degree of matching between the query and the input text; c) for each concept for which enough of the queries have their words in the input text sufficiently close together, calculating a score depending on the sub-scores; and d) determining which of the concepts, for which a score was calculated, pertain to the input text and which do not, depending on the score of the concept.

### RELATED APPLICATIONS

The present application claims the benefit of U.S. Provisional application U.S. Ser. No. 62/771,308, provisionally filed on Nov. 26, 2018, entitled “SYSTEM AND METHOD FOR MATCHING MEDICAL CONCEPTS IN RADIOLOGICAL REPORTS”, in the name of Or Almer, which is incorporated herein in its entirety.

### TECHNICAL FIELD

The disclosure relates generally to the field of natural language processing, and in particular to identifying which medical concepts, from a predefined set of medical concepts, are found in a medical report. More specifically, but not exclusively, the disclosure relates to a method for doing this with radiological reports.

### BACKGROUND

A number of search algorithms exist for automatically determining whether or not a given concept is found in a given input text. For example, the concept could be a search term, a string of words entered into a search engine, and the input text could be text found on one of a large number of web pages that the search engine is searching. In some search algorithms, the search term need not be found in exactly the same form in the input text, in order for the search algorithm to return a positive result, but some of the words in the search term might be missing from the input text, or the words of the search term might be found in a different order, and/or with other words between them, in the input text. In such a case, the search algorithm may calculate a score for that search term and input text, indicating how good the match is, and when the search engine is finished searching the web pages that it is searching, it may provide a list of the web pages for which a positive result was found for that search term, ranked in order of the score.

For example, ElasticSearch is a commercially available search engine described at <https://www(dot)elastic(dot)co/products/elasticsearch>, with a user guide found at <https://www(dot)elastic(dot)co/guide/en/elasticsearch/guide/current/index(dot) html>. ElasticSearch analyzes text fields, such as the title fields of documents, by using different types of queries to calculate a relevance score of a document to the search term. In a “match” query, the text field is considered a match to the search term if more than a minimum number or more than a minimum percentage of words in the search term are found in the text field, and the “match” relevance score is higher if more of the words in the search term are found in the text field. The “match” query of ElasticSearch is an example of a “bag of words” method in which the order of words in the input text field does not matter.

In a “match_phrase” query in ElasticSearch, the text field is considered a match to the search term if all of the words of the search term are found in the text field, and if the relative positions of the words in the search term are not too far apart from the relative positions of the same words in the text field. How far apart the positions of the words are means how many changes must be made in the positions of the words in the search term, each time changing the position of a word by 1 in either direction, in order to make the words have the same positions as they have in the text field. For example, if the search term consists of two words, and those two words are found adjacent to each other but in reverse order in the text field, then the number of changes in position is 2, because each of the two words must undergo one change in position. If the two words are found in the same order as in the search term but with n other words between them in the text field, then the required number of changes in position is n. In order for the text field to be considered a match to the search term, the required number of changes in position must be no greater than a maximum number, a parameter called “slop.” The “match phrase” query may have a higher relevance score if the required number of changes in position is smaller. The “match” relevance score and the “match_phrase” relevance score may be added together to obtain an overall relevance score of the text field to the search term.

It is known for search algorithms to replace words in the text field and/or in the search term with their stems. For example, ElasticSearch has an English stemming feature that does this for English text.

Savova et al, “Mayo clinical Text Analysis and Knowledge Extraction System (cTAKES): architecture, component evaluation and applications,” J. Am. Med. Inform. Assoc. 2010: 17: 507-513 (doi:10.1136/jamia.2009.001560), describes the cTAKES system for extracting information from electronic medical record clinical free-text, and provides a history of other systems that have this goal.

The SNOMED CT medical dictionary, found at <https://browser(dot)ihtsdotools(dot)org>, has a structured list of a large number of medical concepts, including body parts, diseases, and symptoms that can be used to diagnose diseases. Each concept is defined by a string of one or more words. In some cases multiple concepts refer to the same thing, in which case a preferred term and alternative terms are given. Concepts may have parents and children, referring to more general and more specific concepts, for example lung is a parent of lung part, which is a parent of lobe of lung. Disease concepts have a finding site relationship with a body part concept, for example lung cancer has a finding site relationship with lung.

### SUMMARY

An aspect of some embodiments of the invention concerns a method for determining which of a set of medical concepts pertain to an input text from a medical report, in which a concept is considered a possible match to the input text if a sufficiently large number of pairs of words in the concept are both found in the input text sufficiently close to each other.

According to one aspect of the disclosure, there is provided a method of determining which concepts in a set of medical concepts pertain to an input text, automatically by executing instructions on a computer, the method comprising:
a) creating a set of one or more queries for each concept that has two or more words, each query being a string of two words that is a sub-string of the words in the concept, in the same order as the words in the concept;
b) for each concept in a selected sub-set of the concepts that have two or more words, for each query, determining whether or not the input text includes all the words of that query, and if it does, calculating a sub-score indicating a degree of matching between the query and the input text;
c) for each concept in the selected sub-set, for which more than a minimum number of the queries have all of their words in the input text sufficiently close together according to a criterion, calculating a score to indicate a degree of matching between the concept and the input text, depending on the sub-scores of the queries for that concept; and
d) applying one or more rules to determine which of the concepts in the selected sub-set, for which a score was calculated, pertain to the input text and which do not, at least some of the rules depending on the score of the concept.

Optionally, the method also comprises calculating a match score at least for each concept in the selected sub-set that has two or more words, of which more than a minimum number of the words are in the input text, and for which more than the minimum number of the queries have all of their words in the input text sufficiently close together according to the criterion, the match score indicating a degree of matching between the concept and the input text according to a bag-of-words method, and wherein calculating the score for each concept comprises calculating the score depending on the match score for that concept as well as on the sub-scores of the queries for that concept.

Optionally, calculating the score for each concept comprises calculating a weighted sum of the match score and the sub-scores for the queries.

Optionally, the minimum number of words is 2 for concepts with two words, 2 or 3 for concepts with three words, 2, 3 or 4 for concepts with four words, 3 or 4 for concepts with five words, and 3, 4 or 5 for concepts with six words.

Optionally, the method also comprises assigning a score to the concepts that have only one word, when the one word is found in the input text, wherein the rules that depend on the score of the concept are applied both to the concepts with only one word and to the concepts with two or more words.

Optionally, the one or more rules specify that when two concepts for which scores have been calculated have sufficiently great overlap in their words, then the concept with a lower score does not pertain to the input text, and that a concept does pertain to the input text if it has a calculated score that is higher than the score calculated for any other concept with which it has sufficiently great overlap in its words.

Optionally, the first minimum number is between 35% and 65% of the number of queries created for that concept.

Optionally, calculating the score comprises calculating a weighted sum of the sub-scores of the queries, with lower weight given to queries with words that are further apart in the concept.

Optionally, the selected sub-set of concepts excludes at least those concepts for which one or more words defined as mandatory words for that concept are not found in the input text.

Optionally, the word in the concept that is rarest among all the words in the set of concepts is defined as a mandatory word.

Optionally, any singular word in the concept that is a name of a disease is defined as a mandatory word.

Optionally, the selected sub-set of concepts excludes at least those concepts which include a word for a body part and a word describing a location or direction of the body part, for which the word that describes the location or direction of a body part is more than one word away in the input text from the word for the body part.

Optionally, the criterion for words in the query being sufficiently close together in the input text specifies a maximum distance between the words that is lower for words that are not in the same order in the input text as they are in the query, and that is higher for words that are spaced further apart in the concept than for words that are spaced closer together in the concept.

Optionally, the maximum distance for words that are adjacent in the concept and are in the same order in the query and in the input text is between 10 and 25. Optionally, the method also comprises preparing the set of concepts and preprocessing the input text, comprising:
a) providing an initial set of concepts from a database of medical concepts;
b) modifying the initial set of concepts by expanding vertebrae letter-number designations to include the words “vertebra” and “spine” and replacing the letter of the letter-number designation by the body region that it stands for, cervical, thoracic, or lumbar; and
c) preprocessing the input text by expanding vertebrae letter-number designations to include the words “vertebra” and “spine” and replacing the letter of the letter-number designation by the body region that it stands for, cervical, thoracic, or lumbar.

According to another aspect of the disclosure, there is provided a computer storage product having at least one computer storage medium having instructions stored therein causing one or more computers to perform an exemplary method of the invention.

According to another aspect of the disclosure, there is provided a computer storage medium having instructions stored therein for causing a computer to perform an exemplary method of the invention.

According to another aspect of the disclosure, there is provided a computer product embodied in a computer readable medium for performing the steps of an exemplary method of the invention.

According to another aspect of the disclosure, there is provided a system for automatically identifying which concepts in a set of medical concepts are found in a medical report, the system comprising:
a) a first database that provides access to one or more medical reports;
b) a splitter module with access to the first database that divides a medical report into input texts;
c) a second database that provides access to the set of medical concepts;
d) a processor module, with access to the input texts and to the second database, configured, for each input text, to:
   1) create a set of one or more queries for each concept that has two or more words, each query being a string of two words that is a sub-string of the words in the concept, in the same order as the words in the concept;
   2) for each concept in a selected sub-set of the concepts that have two or more words, for each query, determine whether or not the input text includes all the words of that query, and if it does, calculate a sub-score indicating a degree of matching between the query and the input text;
   3) for each concept in the selected sub-set, for which more than a minimum number of the queries have all of their words in the input text sufficiently close together according to a criterion, calculate a score to indicate a degree of matching between the concept and the input text, depending on the sub-scores of the queries for that concept; and
   4) apply one or more rules to determine which of the concepts in the selected sub-set, for which a score was calculated, pertain to the input text and which do not, at least some of the rules depending on the score of the concept; and
e) an output module that outputs the concepts that are determined to pertain to the input texts of the medical report.

According to another aspect of the disclosure, there is provided a method of determining which concepts in a set of medical concepts pertain to an input text, automatically by executing instructions on a computer, the method comprising:
a) for each concept in the set, applying one or more criteria for determining whether of not the concept is a possible match for the input text, wherein, for at least some of the concepts, the criteria do not require that all of the words of the concept are found in the input text, but do require that one or more words specified to be mandatory words of the concept are found in the input text; and
b) applying one or more rules to determine which of the concepts that are possible matches to the input text pertain to the input text, and which do not.

Optionally, the method also includes calculating a score, for each concept that is a possible match to the input text, indicating a degree of matching between the concept and the input text, wherein at least some of the one or more rules depend on the score of the concept and the scores of any other concepts that are possible matches to the input text.

Optionally, a word of the concept that is least common among all the concepts in the set is specified to be a mandatory word for that concept.

Optionally, a word of a concept that is a one-word term for a disease is specified to be a mandatory word for that concept.

Optionally, for a concept that includes a word for a body part and a word that describes a location of direction of the body part, both words are specified to be mandatory words for that concept, and the criteria further require that the words are adjacent to each other in the input text.

According to another aspect of the disclosure, there is provided a method of determining which concepts in a set of medical concepts pertain to an input text, automatically by executing instructions on a computer, the method comprising:
a) for each concept in the set, applying criteria for determining whether or not the concept is a possible match for the input text;
b) for each concept that is a possible match to the input text, calculating a score indicating a degree of matching between the concept and the input text; and
c) determining that, when two concepts for which scores have been calculated have sufficiently great overlap in their words, then the concept with a lower score does not pertain to the input text, and that a concept does pertain to the input text if it has a calculated score that is higher than the score calculated for any other concept with which it has sufficiently great overlap in its words.

Optionally, for at least some of the concepts in the set, calculating the score comprises calculating a match score, calculating a match phrase score, and combining the match score and the match phrase score to obtain the score, the match score depending on how many of the words in the concept are found in the input text but not on the order of those words in the input text, and the match phrase score depending both on how many of the words in the concept are found in the input text, and on the order of those words in the input text.

According to another aspect of the disclosure, there is provided a system for automatically identifying which concepts in a set of medical concepts are found in a medical report, the system comprising:
a) a first database that provides access to one or more medical reports;
b) a splitter module with access to the first database that divides a medical report into input texts;
c) a second database that provides access to the set of medical concepts;
d) a processor module, with access to the input texts and to the second database, configured, for each input text, to:
   1) for each concept in the set, applying one or more criteria for determining whether of not the concept is a possible match for the input text, wherein, for at least some of the concepts, the criteria do not require that all of the words of the concept are found in the input text, but do require that one or more words specified to be mandatory words of the concept are found in the input text; and
   2) applying one or more rules to determine which of the concepts that are possible matches to the input text pertain to the input text, and which do not; and
e) an output module that outputs the concepts that are determined to pertain to the input texts of the medical report.

According to another aspect of the disclosure, there is provided a system for automatically identifying which concepts in a set of medical concepts are found in a medical report, the system comprising:
a) a first database that provides access to one or more medical reports;
b) a splitter module with access to the first database that divides a medical report into input texts;
c) a second database that provides access to the set of medical concepts;
d) a processor module, with access to the input texts and to the second database, configured, for each input text, to:
   1) for each concept in the set, applying criteria for determining whether or not the concept is a possible match for the input text;
   2) for each concept that is a possible match to the input text, calculating a score indicating a degree of matching between the concept and the input text; and
   3) determining that, when two concepts for which scores have been calculated have sufficiently great overlap in their words, then the concept with a lower score does not pertain to the input text, and that a concept does pertain to the input text if it has a calculated score that is higher than the score calculated for any other concept with which it has sufficiently great overlap in its words; and
e) an output module that outputs the concepts that are determined to pertain to the input texts of the medical report.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A shows a block diagram for a system for identifying medical concepts in medical reports, according to an exemplary embodiment of the invention;

FIG. 1B shows a high level flowchart of a method used by the processor module of the system of FIG. 1A, according to any exemplary embodiment of the invention;

FIG. 2 shows a more detailed flowchart of the method used by the processor module of the system of FIG. 1A, according to an exemplary embodiment of the invention; and

FIGS. 3, 4 and 5 show high level flowcharts of methods used by the processor module of the system of FIG. 1A, according to different exemplary embodiments of the invention.

### DETAILED DESCRIPTION OF THE EMBODIMENTS

The following is a detailed description of the preferred embodiments, reference being made to the drawings in which the same reference numerals identify the same elements of structure in each of the several figures.

The disclosure relates generally to the field of natural language processing, and in particular to identifying which medical concepts, from a predefined set of medical concepts, are found in a medical report. More specifically, but not exclusively, the disclosure relates to a method for doing this with radiological reports.

The systems (including computer systems for processing medical reports) and/or methods described herein (e.g., code instructions executed by one or more processors) address the technical problem of automatically identifying the medical concepts that are mentioned in a medical report on a patient, with fewer missed concepts, and fewer false positives, than prior art methods of automatically identifying medical concepts in a medical report. The systems and methods solve this problem by one or more of: 1) not requiring all words of a concept to be present in an input text in order to consider the concept a possible match, even when doing a “match phrase” test where word order matters; 2) requiring certain mandatory words in a concept to be present in the input text, in order to consider the concept a possible match, even if all words do not have to be present; and 3) calculating a score for each concept that is a possible match for the input text, and eliminating a concept as a possible match if there is another concept, that significantly overlaps that concept, that has a higher score.

The systems (including computer systems for processing medical reports) and/or methods described herein (e.g., code instructions executed by one or more processors) improve an underlying technical process within the technical field of natural language processing. The systems and/or methods described herein improve the process of identifying medical concepts in a medical report, by one or more of 1) not requiring all words of a concept to be present in an input text in order to consider the concept a possible match; 2) requiring certain mandatory words to be present; and 3) calculating a score for each possible match and eliminating possible matches when they significantly overlap with a higher scoring possible concept.

The systems (including computer systems for processing medical reports) and/or methods described herein (e.g., code instructions executed by one or more processors) improve performance of a computing unit executing the code instructions that identify medical concepts in medical reports. The improvement in performance increases the number of medical concepts, actually present in each sentence of a medical report, that are successfully identified, while decreasing the number of false positives, medical concepts that are incorrectly identified as being found in sentences of a medical report. The improvement in performance is achieved at least in part by not requiring all words of a concept to be present in an input text, which increases the number of concepts successfully found, by requiring certain mandatory words of the concept to be present, which decreases false positives due to bad guessing of missing words, and by finding a score for each possible concept and eliminating possible concepts that significantly overlap with other higher scoring possible concepts, which decreases false positives due to identifying broader concepts when only narrower concepts are present.

The systems (including computer systems for processing medical reports) and/or methods described herein (e.g., code instructions executed by one or more processors) are tied to physical real-life components, because they process medical reports stored in data storage media, and because the medical reports are written by doctors to describe medical tests performed on real patients, using medical diagnostic equipment, for example medical imaging equipment such as CT scanning devices and MRI devices. The system and methods improve patient care, by making it possible to retrieve medical information from the medical reports much sooner than if the medical concepts had to be identified manually by a human reader of the medical reports, and more accurately than prior art computer systems that identify medical concepts in medical reports automatically.

The systems (including computer systems for processing medical reports) and/or methods described herein (e.g., code instructions executed by one or more processors) provide a unique, particular, and advanced technique of identifying medical concepts in medical reports.

Accordingly, the systems and/or methods described herein are inextricably tied to computer technology and physical hardware, to overcome an actual technical problem arising in natural language processing of medical reports.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s).

In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

An object of some embodiments of the present disclosure is to automatically identify the medical concepts mentioned in the reports that doctors write up after examining patients, selected from a comprehensive database of medical concepts, using a computer system performing natural language processing. In some embodiments, the computer has better performance than computers using existing methods for that purpose, both in identifying a higher percentage of the concepts that are mentioned in the report, and in not mistakenly identifying as many medical concepts that are not found in the report. Simultaneously achieving improved performance by both of these measures is challenging, since doctors often leave out words of a concept that they believe that another doctor reading the report will understand are implied. Finding those concepts requires guessing the words that are left out, but guessing words can also result in mistakenly identifying more concepts that are not really found in the report. A useful measure of success in achieving these objects is the parameter F1, which is the harmonic mean of the percent of concepts present that are identified, and the percent of identified concepts that are really present.

Another object of some embodiments of the present disclosure is to achieve such an improved performance, for example as measured by F1, while processing medical reports quickly enough so that the method can serve as a first stage of a larger natural language processing system for radiological reports. For example, in some embodiments the computer can process at least 100 reports per second, using an Intel® Core™ i7-6700 CPU at 3.40 GHz with 4 cores and 8 logical processors.

Another object of some embodiments of the present disclosure is to achieve such an improved performance without the algorithm having a deep AI-like understanding of each concept. Providing such a deep AI-like understanding of each concept could make development of the algorithm very labor intensive.

These objects are given only by way of illustrative example, and such objects may be exemplary of one or more embodiments of the invention. Other desirable objectives and advantages inherently achieved by the may occur or become apparent to those skilled in the art. The invention is defined by the appended claims.

Useful features of the computer system

An aspect of some embodiments of the invention concerns a computer system that determines which of a set of medical concepts is found in a medical report, for example a radiology report, automatically by executing instructions on the computer, with one or more of three features that the inventor has found are often effective at reducing false positives, and at not missing too many concepts that are really present. These features can be explained in terms of two approaches to the problem of automatically identifying concepts in a report, using a computer. The bag-of-words method, as exemplified by the match test in ElasticSearch, considers a concept to match an input text, for example one sentence of the medical report, if all the words of the concept, or almost all the words, are found in the input text, regardless of word order. This method tends to be fairly efficient at finding the concepts that are present, but tends to produce a lot of false positives, finding concepts that are not really present. Another method, used by the cTAKES algorithm and exemplified by the match phrase test in ElasticSearch, considers a concept to match an input text if all the words of the concept are found in the input text in the same order, or almost the same order, as in the concept. This method tends to be somewhat better than the bag-of-words method at eliminating false positives, but somewhat worse at finding the concepts that are present. The inventor has found that in some embodiments of the invention, a method employing any one or two of the following features, or better yet all three of the following features, can give better results both in eliminating false positives and in finding the concepts that are really present:
1) Not requiring all words to be present even in the match phrase test, where word order matters. This allows additional concepts to be found, when the doctor who wrote the report omitted words from the concept that they thought would be obvious to human readers of the report.
2) Requiring certain mandatory words to be present, whether in the match test or the match phrase test, even if all words do not have to be present. This reduces the number of false positives that would result from guessing important words that generally would not have been omitted by the doctor who wrote the report.
3) Calculating a score from the match test, or the match phrase test, or better yet from a combination of both tests, and using the score to eliminate concepts that have lower score, when there is another concept, which significantly overlaps that concept, with a higher score. This reduces false positives, by excluding related broader concepts, when only the narrower concept is present, as well as excluding concepts with missing words when a similar concept without missing words, or with fewer missing words, is present.

The inventor has found, in some cases, that an algorithm that includes all three of these features produces fewer false positives, and finds a larger percentage of the concepts that are really present, than either the match test or the match phrase test by itself.

The first feature, not requiring all words of the concept to be present in the match phrase test, is optionally implemented as follows. Each concept is a string of words. For each concept of two words or more, one or more queries are generated, each query consisting of a 2-word sub-string of the words of the concept, optionally in the same order as in the concept. The query need not be a proper sub-string of the words of the concept, but in the case of a 2-word concept, the query could be identical to the concept. The concept is optionally considered a possible match to the input text if at least a minimum number of the queries have both their words in the input text, sufficiently close together. Optionally the minimum number depends on the total number of queries for that concept, for example the minimum number is a percentage of the total number of queries, for example 35% of the queries, or 40%, 45%, 50%, 55%, 60%, or 65%. Optionally there may also be other criteria that have to be satisfied, in order to consider the concept as a possible match to the input text, for example requiring mandatory words to be present. If the concept is considered a possible match to the input text, then a query sub-score is found for each query, the query sub-score being higher if the two words of the query are found closer together in the input text, and if the two words are in the same order in the input text as in the concept. An overall match phrase score for the concept, based on the query sub-scores, for example a weighted average of the query sub-scores, is then found, and a concept is not considered to pertain to the input text if it has sufficient overlap with another concept that has a higher score. Optionally, the weighted average gives lower weight to queries for which the two words are further apart in the concept.

The second feature, requiring certain mandatory words to be present in the input text, is optionally implemented as follows. Optionally, the word in the concept that is the least common of the words in the concept, among the words of all of the concepts in the set of concepts, is a mandatory word that must be found in the input text. Optionally, any singular word that is the name of a disease is a mandatory word that must be found in the input text. Optionally any word referring to a direction or a location of a body part must be adjacent, in the input text, to the word for the body part that it refers to, in order to consider the concept as a possible match to the input text.

Optionally, in addition to finding which concepts are possible matches to the input text by seeing which queries are found in the input text, and what their query sub-scores are, concepts are also tested to see if they are possible matches to the input text according to a bag-of-words method, referred to as a match test. In a match test, a concept is considered a possible match if a sufficient number of words in the concept are found in the input text, where the minimum number of words may depend on the number of words in the concept. If a concept is considered a possible match to the input text according to this criterion, then optionally a match score is found for the concept, optionally depending only on the number of words of the concept that are present in the input text, regardless of the order of the words in the input text.

Optionally, the match score is combined with the match phrase score described above, for example a weighted sum of the match score and the match phrase score is found, to find a total score for the concept, and it is the total score that is used to decide which of two concepts to keep, when they have sufficient overlap. Optionally, a concept is considered a possible match to the input text only if it is considered a possible match both according to the criterion of having enough of the queries match the input text (the “match phrase test”), and according to the criterion of having enough of the words of the concept present in the input text (the “match test”). Requiring a concept to pass both the match test and the match phrase test may make it more likely that a concept pertains to the input text even if some of the words of the concept are not found in the input text. Alternatively, only the match test is done, or only the match phrase test is done, or both tests are done but the concept only has to pass one of the tests to be considered a possible match to the input text.

Optionally, the match test is applied also to one word concepts, for which a match phrase test is not applied, and a match phrase score is not calculated, because there are no two-word queries in a one word concept. A one word concept passes the match test if the one word is found in the input text, and optionally its match score, and its total score, is always the same. Optionally, for a one word concept, the match phrase score is zero. Optionally, one word concepts have their scores compared to other multi-word concepts that overlap them, for example to other concepts that also have that word, and that pass the match test and the match phrase test. Optionally, the score of the multi-word concept will always be higher in this case, and the one word concept will be eliminated from consideration.

Optionally, the set of concepts is preprocessed by stemming the words, i.e. replacing words by their stems. Optionally, stop words are removed from the concepts, in the pre-processing. Optionally, words in the concepts are replaced by a preferred synonym, when several medical terms have the same meaning. For example, an adjective such as “hepatic” is replaced by a corresponding noun, in this case “liver.” Optionally, acronyms in the concepts are expanded. Optionally, vertebrae referred to by the letter-number designation have the words “vertebra” and “spine” added to them, and the letter is replaced by the region of the spine that it refers to, either “cervical,” “thoracic” or “lumbar”. Additionally or alternatively, any or all of the preprocessing procedures described herein for the concepts are applied to the input text.

It should be noted that the methods described and claimed herein are not directed to abstract ideas, but to methods of improving computer technology for natural language processing, and the claims do not pre-empt all methods of identifying medical concepts in a medical report, but are limited to particular methods of achieving that result. For example, at least some of the claims may be limited to a method where two-word queries are created from the concepts, and performing a match phrase test for each concept by seeing how many of the two-word queries are a match to the input text. At least some of the claims may be limited to methods where certain mandatory words of the concept must be present in the input text, in order for the concept to be considered a possible match for the input text. These elements of the methods, which are believed to be novel and inventive over the prior art, are suited specifically for using computer technology to identify medical concepts found in a medical report, and are very different from the methods that a person, using the natural language processing ability of the human brain, would use for that purpose.

### Description of the System

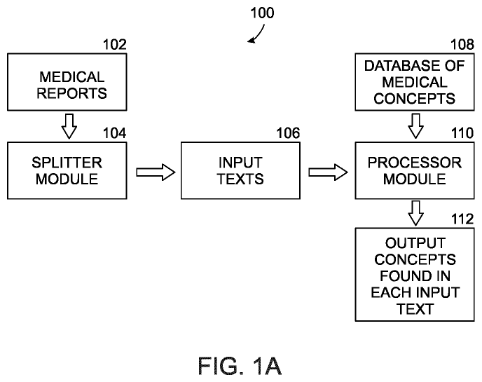

Referring now to the drawings, FIG. 1A illustrates a block diagram 100 for a system that implements the method according to an exemplary embodiment of the invention. Each block in diagram 100 represents a software module, as stored in a computer that is programmed to implement the module, or a server that stores or provides access to data, for example in digital form. A report database 102 stores or provides access to medical reports that the system will process. A splitter module 104 divides each report into multiple input texts 106, each of which is processed separately to find the concepts it contains. Typically each sentence of a report is a separate input text. Optionally, long sentences, especially compound sentences, are divided into more than one input text, for example by a parser that uses grammatical rules to attempt to determine the boundaries of each component of the compound sentence. In the tests done by the inventor, long sentences are divided by the splitter provided by the Natural Language Tool Kit in Python, but alternatively any other sentence splitter known in the art can be used. Optionally, sentences are split at isolated commas, for example commas that have no other comma within 15 characters of them, but not at commas that do have another comma within 15 characters, because those commas are likely to be part of a list, such as in “lung, liver or throat cancer,” that bridge a single concept.

A concept database 108 has a list of medical concepts, for example the medical concepts listed in the SNOMED database, or a selection of concepts listed in the SNOMED database. Optionally, concept database 108 does not include all of the concepts listed in SNOMED or a similar database, but is limited to concepts describing disorders and morphological abnormalities, and is limited to concepts that are not longer than a maximum number of words. For example, the inventors have generally used a database of concepts limited to concepts which have 6 words or less, after preprocessing, since the method tends to have a lower success rate for concepts with more words, and such concepts are found less frequently in medical reports. In the current implementation of the concept database, only concepts that describe disorders and morphological abnormalities are included. There are a total of 107,453 concepts, which, after preprocessing, include 4,606 one-word concepts, 24,399 two-word concepts, 25,838 three-word concepts, 22,815 four-word concepts, 18,329 five-word concepts, and 11,466 six-word concepts.

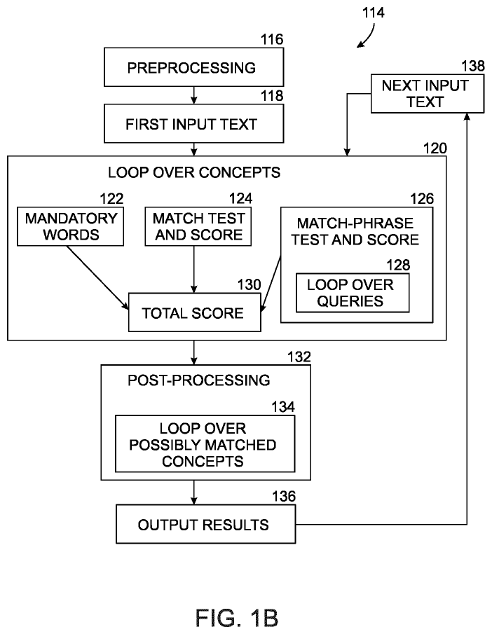

A processor module 110, with access to both the input texts 106 and the list of medical concepts 108, uses an algorithm to identify the medical concepts found in each input text, as will be described in FIG. 2, though in general this process produces some errors. The results of this process, a list of medical concepts found in each input text, are optionally sent to output module 112, which outputs them in a form that a user can read. Optionally, output module does not separately list, in the output, the medical concepts found in each input text, but only collectively lists the medical concepts found in a set of input texts, for example in the entire medical report.

### Overview of Method

The functioning of processor module 110 in FIG. 1 is described in more detail by flowchart 200 of FIG. 2. An overview of flowchart 200, showing the different parts of the algorithm at a high level, is shown in flowchart 114 of FIG. 1B.

At 116 of FIG. 1B, preprocessing of the concepts and the input texts. Preprocessing of the concepts need only be done once before processing many input texts, and optionally is only done again if the set of concepts is being revised. Preprocessing of the input text is optionally done for each new input text that is being processed, but could be done for a set of input texts, for example for all the input texts of a medical report, before processing any of them. Preprocessing of both concepts and input text includes, for example, stemming, removing stop words, expanding acronyms, and replacing words with synonyms. Preprocessing of concepts may also include, for example, generating queries for concepts of two or more words, finding mandatory words for each concept, and eliminating certain concepts, for example concepts that have more than a maximum number of words.

At 118, the first input text is considered.

At 120, a loop is performed over concepts, for the input text being considered. Loop 120, and each of the other loops shown in FIG. 1B, may be performed in series or in parallel, or using a combination of series and parallel processing. For the detailed flowchart shown in FIG. 2, these loops are all shown as being processed in series. Loop 120 includes, for each concept: 1) checking the input text for mandatory words at 122; 2) performing a match test and finding a match score at 124; and 3) performing a match_phrase test and finding a match_phrase score at 126. These three tests may be performed in series or in parallel, and if they are performed in series, they may be performed in any order. For the detailed flowchart shown in FIG. 2, these tests are shown as performed in series, first the check for mandatory words, then the match test, and then the match_phrase test. Performing the tests in this order has the potential advantage that the check for mandatory words is generally faster than the match test which is generally faster than the match_phrase test, and the slower tests can be skipped, saving computer time, if the concept does not pass one of the faster tests. Performing the match_phrase test and finding the match_phrase score includes a loop 128 over the queries for that concept. At the end of loop 120, a total score for the concept is found at 130, for example by combining the match score and the match_phrase score. Optionally, in order to be considered as a possible match for the input text, a concept has to pass all three tests, the check for mandatory words at 122, the match test at 124, and the match_phrase test at 126. The tests can be done in any order, or in parallel, but if the tests are done serially, then if a concept fails to pass one of the tests, the loop optionally ends for that concept and goes on to consider the next concept. For the detailed flowchart shown in FIG. 2, the check for mandatory words is shown as done first, followed by the match_test, and the match_phrase test is shown as done last. A record is kept of which concepts pass all the tests and are thus possible matches for that input text, and of the total score for each concept that is a possible match.

After loop 120 has found all of the concepts that are possible matches for that input text, post-processing is done at 132. Post-processing includes a loop 134 over the possible matched concepts. In loop 134, possible matched concepts are eliminated from consideration if they have significant overlap with another possible matched concept that has a higher score, for example because more words of the other concept are found in the input text, and/or the order of words in the input text is closer to the order of words in the other concept.

After post-processing 132 has been completed for all the possible matched concepts, the remaining matched concepts, that have not been eliminated, are outputted at 136. These are the concepts that the algorithm has identified as being found in the input text. Optionally, instead of separately listing the concepts identified in each input text, a list of all the concepts identified in several input texts, for example in all of the input texts in the report, is provided as output after those input texts have been processed.

At 138, the next input text is considered, if there are any remaining input texts that have not been processed.

### Preprocessing of Input Texts and Concepts

At 202, the input texts are preprocessed. Preprocessing optionally includes one or more of the following:

1) Removing stop words, including articles, conjunctions, prepositions, and/or other common words that are not likely to provide information on whether a given concept is found in the input text.

2) Removing ambiguous words that have more than one meaning that are likely to cause errors. For example, the word “exposure” may occasionally refer to a patient being exposed to the cold, which is a medical concept, but it is also used in almost all radiological reports to refer to the exposure parameters of the image. The inventors have found that leaving it out results in a better F1 score for the algorithm. Another example of an ambiguous word, which is not removed in the current implementation of the algorithm but which sometimes causes errors, is “sinus,” which can refer both to a body part and to a condition.

3) Replacing words by their stems, which may be referred to as “stemming.”

4) Replacing all words in a group of words with equivalent meanings by a single preferred form. For example, an adjective like “hepatic” is replaced by the corresponding noun “liver.”

5) Expanding acronyms into the words they stand for. The software optionally includes a list of acronyms used in medical reports, and their expanded forms. Such a list can be created manually, for example. From time to time the list of acronyms is optionally modified, for example by adding new acronyms that have started to appear in medical reports, or by removing an acronym that has been found to be ambiguous, and/or is no longer is common use.

6) For letter-number designations of vertebrae, adding the words “vertebra” and “spine,” and replacing the letter C by “cervical,” the letter T by “thoracic,” and the letter L by “lumbar”.

At 204, the concepts are preprocessed, optionally using the same procedures as described for the input texts. Optionally, concepts that have more than a maximum number of words after preprocessing are removed from the list of concepts. In the current implementation of the algorithm, concepts with more than six words are excluded. Alternatively, concepts with more than five words, or more than seven words, or more than eight words, are excluded. Tests done by the inventor have shown that it is more difficult to determine if a concept is present in the input text, the more words the concept has, and furthermore concepts with a very large number of words are likely to be narrow in scope and not to occur very often, so in practice it is not so important to consider them. At 206, queries are generated for each concept. Optionally, each query consists of a pair of words found in the (preprocessed) concept, in the same order as the words are found in the concept, and there is a query generated for each such pair of words. The “Percolator” feature of ElasticSearch provides a convenient way to generate the queries. It should be understood that the order of words in the query is important, not because the words of the input text are required to be in the same order, but because a concept for which the words in the input text are out of order will generally have a lower score than if they are in the same order as in the concept. Optionally, queries with more than two words are also generated, for example queries with up to three words. However, the inventor has found that there is little to be gained by using queries of more than two words.

At 208, each concept is examined to find the mandatory words it contains. The mandatory words for a concept are words that must appear in the input text, in order to consider that concept a match for the input text. The mandatory words optionally include any of the following:

1) The word, among all the words in that concept, that is the least common word among all the concepts in the set of concepts.

2) Any single word for a disease.

3) Any word indicating a direction or a side of the body, that describes a body part, for example the word “left” in the concept “left lung.” The word indicating direction must be found in the input text within one word of the body part that it describes, in order for the concept to be considered a match to the input text.

In addition, other mandatory words are optionally designated manually for some concepts, for example if they are found to be useful.

It should be understood that procedures 204, 206 and 208 may be done once, before processing any medical reports, and they become part of the version of the algorithm that is being used. They need not be done again for each new report that is processed. When a new version of the algorithm is instituted, there may be some changes in the list of concepts, for example if there is a different choice for words that are to be removed from the concepts, or an expanded database is used to produce the list of concepts, and in that case procedures 204, 206 and 208 may be done over again, at least for the concepts that are new or have been changed.

### Loop Over Input Texts and Loop Over Concepts

At 210, the first input text is considered. At 212, the first concept is considered, to decide whether it is a match for that input text. At 214, it is determined whether the mandatory words for that concept are found in the input text, and at the required positions in the case of any mandatory words, such as direction words describing body parts, that may have required positions.

If all the mandatory words are present, then at 216 it is determined whether the match test is passed. The match test requires that a certain minimum number of words, out of all the words in the concept, be present in the input text, but their order does not matter, because the match test is a “bag of words” test. It should be understood that the number of words present in the input text, and the total number of words in the concept, optionally both refer to after the concept and input text have been preprocessed, for example removing stop words, and stemming The inventor has found the best results when the minimum number of words in the concept that must be present in the input text to pass the match test is greater than ⅔ of all the words in the concept, but not greater than ¾ of all the words in the concept. But reasonably good results have been obtained by setting the minimum at any value greater than ½ and not greater than ⅚ of all the words in the concept. For example, for two word concepts, optionally both words must be found in the input text. For three word concepts, optionally two words must be found, or alternatively all three words must be found in the input text. For four word concepts, the minimum number of words that must be found in the input text is optionally two, or three, or four. For five word concepts, the minimum number of words is optionally three, or four. For six word concepts, the minimum number of words is optionally three, four, or five. Optionally, the minimum number of words is 50%, or 60%, or 70%, or 80%, of the total number of words in the concept.

If the concept passes the match test, then at 218 the match score is found and recorded. The match score depends on how many words in the concept are found in the input text. If a greater number of words in the concept are found in the input text, then it is generally more likely that the concept is found in the input text, even if the percent of the words of the concept that are found in the input text is not greater. Optionally, the match score depends only on the number of words of the concept found in the input text, without regard to how many words are in the concept. Alternatively, the total number of words in the concept is also taken into account, in calculating the match score. In an exemplary implementation of the method, the match score is the square root of the number of words of the concept that are found in the input text.

### Loop Over Queries for Match Phrase Test

At 220, the first query for that concept is considered, at the beginning of a loop that will look at all the queries for that concept, in order to determine whether the concept passes the match phrase test, and in order to calculate the match phrase score. It should be understood that, in the case of a one word concept, there are no queries, since each query contains two words from the concept, and so no match phrase test can be done, and no match phrase score can be calculated. In that case , although this is not explicitly shown in flowchart 200, control passes to 238, where the next concept is sought.

At 222, it is determined whether the query being considered is found in the input text. That is, it is determined whether both words of the query are found in the input text, not necessarily in the same order as they are found in the query (which means in the same order as in the concept). If queries with more than two words are used, then it is determined whether all the words of the query are found in the input text. However, if the positions of these words in the input text are too different from their positions in the query, then it is considered that the query is not found in the input text.

How different the positions of the words are, is optionally defined by the number of one-step changes in position it would take to make the positions of the words in the query match the positions of the those words in the input text. For example, if the query has two words, and the two words in the input text are adjacent to each other, in the same order as in the query, then the difference s in the positions is zero. If the two words are adjacent to each other in the input text, but in the reverse order of the words in the query, then s=2, because each word in the query would have to move by one position, to be in the same positions as they are in the input text. If the words in the input text are in the same order as the words in the query, but there are n other words between them, then s=n. And if the words in the input text are in the reverse order, and with n other words in between them, then s=n+2.

The parameter specifying the maximum s that is allowed, in order to consider the query to be found in the input text, is referred to as “slop.” The slop value is optionally dependent on how far apart the words of the query are in the concept, with a higher slop value when the words of the query are further apart in the concept. For example, if d is the distance in the concept between the two words in the query, with d =0 for words that are adjacent in the concept, then “slop” is optionally equal to d plus a constant, for example d+15. The inventor has found that the performance of the algorithm is best when “slop” is between approximately d+10 and d+25. In practice, most sentences in medical reports have fewer than 15 words, and for those input texts, the differences in positions never exceeds “slop” if it is defined as d+15, and all queries are counted as being found in the input text, if the words of the query are found in the input text. However, there are some unusually long sentences in medical reports, in which several concepts are often found, and using a value of “slop” between d+10 and d+25 can prevent the algorithm taking one word from one concept and one word from another concept, and inappropriately matching them to a single query. It is because of these relatively few unusually long sentences that the performance of the algorithm depends on the value chosen for “slop.”

At 224, if the query is found in the input text, then the query sub-score is calculated and recorded. The query sub-score is optionally based on how different the positions of the words of the query are in the input score, from their positions in the query, with a lower query sub-score if the positions of the words are more different. Optionally, the difference in positions s is defined as described above. For example, the query sub-score is proportional to 1/(s+1), with s defined as described above. For example, the query sub-score is √2/(s+1).

At 226, it is recorded whether or not the query is found in the input text. At 228 it is determined whether or not there are any further queries for that concept. If there are further queries, then the next query is examined at 230, and control passes again to 222. When there are no more queries remaining to examine in that concept, then control passes to 232, where it is determined whether or not that concept passes the match phrase test. A concept passes the match phrase test if at least a minimum number of the queries in that concept are found in the input text. For example, at least a minimum percentage of the queries for that concept are found in the input text, for example at least 35%, or at least 40%, or at least 45%, or at least 50%, or at least 55%, or at least 60%, or at least 65%, of the concepts are found in the input text.

### Match_Phrase Score

If the concept passes the match phrase test, then at 234 the match phrase score is calculated for that concept. Optionally, the match phrase score is a weighted sum of all the query sub-scores for the queries of that concept that are found in the input text, divided by the number N of queries of that concept. Optionally, a lower weight is used for queries whose words are further apart in the concept. For example, if the query consists of the i^{th} and the j^{th} words in the concept, then the weight used for that query is 1.2-0.15(i−j). Alternatively, the constant 1.2 in this expression is replaced by another constant, for example between 0.9 and 1.5, and/or the constant 0.15 is replaced by another constant between 0.10 and 0.20. It should be noted that, if concepts are only included in the set of concepts if they have 6 or fewer words, then i−j is always between 1 and 5, and the weight 1.2-0.15(i−j) can range from 0.45 to 1.05. Optionally, the expression for weight is chosen so that, given the maximum number of words in the concept, the weight is never negative.

### Total Score for Concept

If the concept passes the match phrase test, then at 236 the concept is recorded as being a possible match for the input text, and the total score for the concept is found and recorded. The total score for the concept depends on both the match score and the match phrase score, for example the total score is a weighted sum of the match score and the match phrase score. For example, the match score is given a weight of 1.5 times the weight of the match phrase score, with the match score defined as the square root of the number of words in the concept that are found in the input text, and the match phrase score defined as

$$\frac{1}{N} {\sum }_{i < j} [ 1.2 - 0.15 ( j - 1 ) ] M P ( i , j )$$

where MP(i, j) is the query sub-score for the query consisting of the i^{th} word and the j^{th} word in the concept, defined as √2/(s+1), with the sum in the expression for the match phrase score being over all the queries in that concept, and N being the number of queries in the concept. It should be understood that the definition of total score will be unchanged if the match score is redefined to differ by a factor x, the match phrase score is defined to differ by a factory, and the relative weights of the match score and the match phrase score are redefined to differ by a factor of y/x. It should also be understood that only the relative weights of the match score and the match phrase matter, if the total score is a weighted sum of the match score and the match phrase score, since the scores are used only to compare the scores of different matched concepts for a given input text, and it makes no difference if all the scores are changed by the same factor.

Once the concept is recorded as being a possible match for the input text, and its total score is recorded, control passes to 238, where it is determined whether there remain any more concepts to be considered for that input text. If all the mandatory words for the concept are not found in the input text, at the required positions, at 214, or if the concept fails the match test at 216, or if the concept fails the match phrase test at 232, then the concept is not recorded as being a possible match for that input text, and control passes directly to 238.

One-Word Concepts

If the concept has only one word, then there are no queries associated with it, since queries each have two words from the concept. In this case, it is not possible to perform a match phrase test or to calculate a match phrase score as described above. Optionally, in this case, a concept is considered a match as long as the match test is passed, which means that the one word of the concept is found in the input text. Optionally, for a one word concept, the total score depends only on the match score, which is always the same for a one word concept, if the match score depends only on the number of words of the concept that are found in the input text. If the match score is defined as the square root of the number of words of the concept found in the input text, then the match score would always be 1 for a one word concept. If the score is defined in general as the weighted sum of the match score and the match phrase score, with a weight of 1.5 for the match score, and if the same definition is used for a one word concept with the match phrase score set to zero, then for a one word concept the total score will always be 1.5, if the concept is a match for that input text. The inventor has found, in some cases, that treating one word concepts in this way results in good performance of the algorithm, finding more of the concepts that are really present, and reducing false positives. Alternatively, a different constant value is used for the total score for all one word concepts for which the one word is found in the input text, for example 1, or 2, or an intermediate value, or a value less than 1 or greater than 2.

If it is determined at 238 that there are more concepts to be considered, in the set of concepts, then the next concept is considered at 240, and control passes again to 214. When there are no more concepts left to consider, then at 242, it is determined whether more than one possibly matching concept has been recorded for that input text. If there is more than one possibly matching concept, then the possibly matching concepts are sorted by score in 244.

Post-Processing of Possibly Matching Concepts

At 246, the second highest scoring possibly matching concept is considered. At 248, the score of the concept being considered is compared to the scores of each of the concepts that have a higher score than it has. The first time this is done, with the second highest scoring concept, there is only one concept with a higher score, that it has to be compared to. If the concept being considered has significant overlap with a concept that has a higher score, then the concept being considered is removed from the list of possibly matched concepts for this input text. Optionally, “significant overlap” is defined as any of these three conditions being true:

1) All of the words in the concept being considered are found in the concept with the higher score.

2) All of the words in the concept with the higher score are found in the concept being considered.

3) The number of words found in one of the two concepts but not in the other concept is sufficiently small relative to the number of words in the concept being considered. For example, the number of words found in one of the concepts but not the other, divided by 1.7, is less than the greatest integer that is less than or equal to 0.5 times the number of words in the concept being considered.

The inventor has found that this definition of “significant overlap” gives good performance of the algorithm. Alternatively, a different definition is used, for example different constants are used instead of 1.7 and/or 0.5, for example a constant between 1.3 and 2.1 is used instead of 1.7 and a constant between 0.35 and 0.65 is used instead of 0.5.

At 250, it is determined whether there are more possibly matching concepts to look at. If there are, then at 252, the next highest scoring concept is considered, and control returns to 248. If there are no more possibly matching concepts to consider, then at 254 the results of the algorithm, the remaining matching concepts for this input text, are listed in the output. These remaining matching concepts are the concepts that the algorithm has found to be present in the input text.

Optionally, the set of concepts includes some groups of two or more concepts that are considered to have the same meaning, and one of the concepts in each group is designated as the more preferred form, and the other concepts in each group are designated as less preferred forms. The concepts are treated as separate concepts throughout the process of the algorithm, until the concepts in the input text have been identified. Then, when the identified concepts are listed in the output, any less preferred forms are replaced by the corresponding more preferred form.

### End of Loop Over Input Texts

At 256, it is determined whether there are any more input texts to process. If there are, then the next input text is looked at, at 258, and control returns to 212, to find the concepts present in the next input text. If there are no more input texts to process, then the algorithm ends at 260.

### Using Only Some Features

As noted above, the inventor has found three features that improve the results over the prior art, allowing the computer system to find more of the concepts present, or to have fewer false positives, or both, compared to the prior art. These features are:

1) not requiring all words of a concept to be present in the input text, even in a match phrase test where word order matters;

2) requiring certain mandatory words of a concept to be present in the input text; and

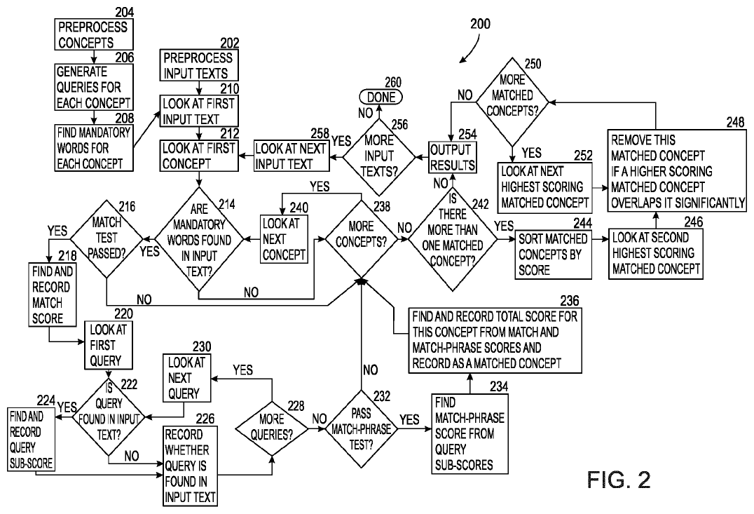

3) calculating a score for each possibly matching concept, and when two possibly matching concepts have significant overlap, eliminating the one with the lower score. Although the inventor has found that the best results are obtained when all three of these features are used, it is also possible to use only one or two of these features. FIGS. 3, 4, and 5 show flowcharts, similar to FIG. 1B, for the cases respectively where only the first feature is used, where only the second feature is used, and where only the third feature is used. In all three cases, the flowchart begins with preprocessing 116, taking the first input text 118, and performing a loop over concepts 120, as described above for FIG. 1B. And in all three cases, the flowchart ends with outputting the results 136 for that input text, and for then looking at the next input text 138, as described above for FIG. 1B. The flowcharts differ in what is included in the loop over concepts, and in whether or not they include post-processing.

In FIG. 3, flowchart 300 shows a method of identifying medical concepts in a medical report, where, in each input text, all the words of a concept do not have to be found in the input text, in a match phrase test, in order for the concept to be considered a possible match to the input text. The loop over concepts 120 shows only a match phrase test 128, though optionally a match test is also done, and optionally mandatory words are checked for. No calculation of scores is shown, and no post-processing is shown, in flowchart 300. The match phrase test optionally includes a loop over queries, with each query consisting of a pair of words from the concept, and all queries need not be found in the input text. For example, the match phrase only requires a certain number of the queries, for example 50% of the queries, to be found in the input text, in order for the concept to pass the match phrase test. Alternatively, the match phrase test does not include a loop over queries, but, for example, the match phrase test requires that a certain number of words of the concept, less than 100% of the words, are found in the input text, in order to pass the match phrase test. Even with only this feature, the computer system may find more of the concepts that are present in the input text, than in prior art where all words in the concept have to be present in the input text, in order to pass a match phrase test.

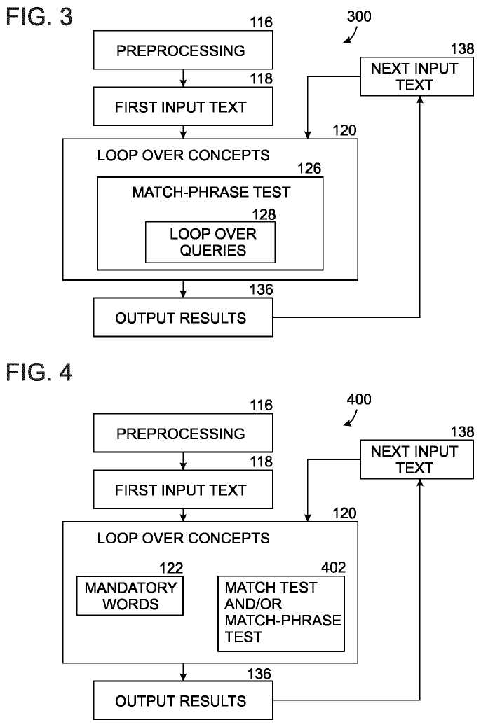

In FIG. 4, a flowchart 400 shows a method of identifying medical concepts in a medical report, where certain mandatory words, for each concept, have to be found in the input text, in order for the concept to be considered a possible match to the input text, even though all words in the concept do not have to be found in the input text. The loop 120 over concepts includes a check 122 that the mandatory words are present. Loop 120 also includes a test 402, for example a match test and/or a match phrase test, which does not always require that all words of the concept be present in the input text, in order to pass the test, but does require that a certain number of words of the concept be present, in order to pass the test. In flowchart 400, as in flowchart 300, no calculation of scores is shown, and no post-processing step is shown.

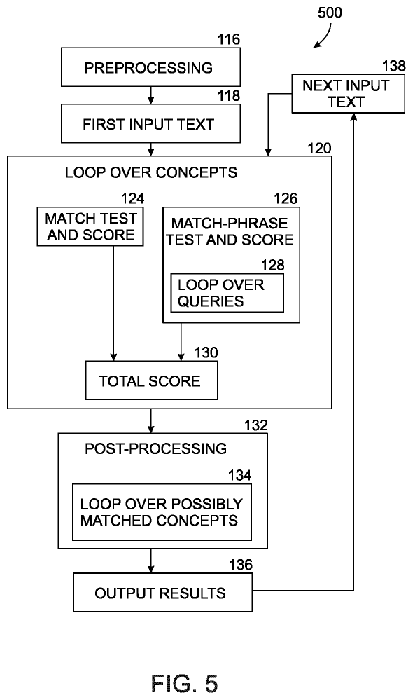

In FIG. 5, a flowchart 500 shows a method of identifying medical concepts in a medical report, in which each possibly matching concept is given a score, indicating a degree of matching to the input text, and lower scoring concepts are excluded as possibly matching, when there is a higher scoring concept that has a significant overlap with the lower scoring concept. Loop 120 over concepts shows both match test 124, and a match phrase test 126, with a score calculated for each one, if the concept passes the test and is found to be possibly matching concept. The match phrase test includes a loop 128 over queries. The match score and the match phrase score are combined, at 130 to obtain a total score, for example by taking a sum or a weighted sum of the match score and the match phrase score. Alternatively, only the match test or only the match phrase test is done in the loop over concepts, and the score is only the match score, or only the match phrase score. Post-processing is done at 132, by performing a loop 134 over possibly matched concepts. For each possibly matched concept, the concept is excluded as a possibly matched concept if there is a higher scoring possibly matched concept that significantly overlaps it. This post-processing prevents broader concepts from being identified in the input text, if only a narrower concept is found. For example in an input text that only mentions the left lung, the concept “left lung” will be identified, but the broader concept “lung” will not be.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

To test the algorithm, and to compare its performance to prior art algorithms that find concepts in medical reports, test data was prepared from 107 radiology reports. Each sentence of each report was manually examined to determine which concepts in a set of concepts, taken from the SNOMED CT medical dictionary, limited to concepts with no more than six words (after preprocessing as described above, to remove stop words, etc.), and limited to concepts that describe disorders and medical abnormalities. In this way, it was determined which concepts were really found in each sentence. A total of 860 concepts were found in the sentences of the 107 reports.

The algorithm was used to identify which concepts in the set occurred in each sentence of the reports. A count was made of the total number of true concepts identified (true positives), the total number of concepts identified that were not really found in that sentence (false positives), the total number of concepts that were really present in each sentence that the algorithm failed to identify (false negatives), and the total number of concepts not present in each sentence, that the algorithm correctly did not idenfity (true negatives). Two quantities were then calculated. Precision is the ratio of true positives to total positivies. This is the fraction of all concepts identified by the algorithm that were really present. Recall is the ratio of true positives to true positives plus false negatives. This is the fraction of all concepts really present that the algorithm identified. The quantity F1 is the harmonic mean of the precision and the recall, which may be defined as 2/(1/precision+1/recall).

The same test was also made with five other methods, some of them known in the prior art. In all these tests, lower scoring concepts were eliminated in favor of higher scoring concepts when there was significant overlap between them, and the same set of concepts were used (the concepts in SNOMED with 6 or fewer words after preprocessing).

1) Match test and match score only (bag of words model), requiring at least 75% of the words in the concept to be present in the input text.

2) An algorithm similar to the cTAKES algorithm, using match phrase with zero “slop,” requiring all words in the concept to be present, in the same order as in the concept and with no other words in between.

3) Match phrase with some “slop,” requiring all words in the concept to be present, not necessarily in order, but not counting a concept as a match if the words in the concept are too far apart or too out of order in the input text. The value of “slop” was between 10 and 15, and the results did not depend very much on the exact value of slop within that range.

4) Hybrid algorithm including both a match score and a match phrase score (with some “slop”) in the score, with the match phrase test requiring all words in the concept to be present in the input text.

5) Hybrid algorithm including both a match score and a match phrase score, where the match phrase score and match phrase test are based on a set of all the two-word queries in the concept, rather than requiring a match with all the words in the concept, but without using mandatory words.

The results of the tests are shown in Table 1. The computer system, using an Intel® Core™ i7-6700 CPU at 3.40 GHz with 4 cores and 8 logical processors, and using the full algorithm, was able to process over 100 radiological reports per second.

TABLE 1
Test results for different algorithms

| | | | | | Hybrid, | |
|---|---|---|---|---|---|---|
| | | Match | Match | Hybrid, | 2-word | |
| | | phrase | phrase | zero slop | queries | |
| | Match | only, zero | with some | in match | in match | Full |
| | only | slop | slop | phrase | phrase | algorithm |
| Precision | 61.6% | 75.7% | 78.3% | 86.5% | 75.1% | 88.3% |
| Recall | 80.8% | 70.2% | 78.7% | 85.9% | 86.7% | 89.7% |
| F1 | 69.9% | 72.9% | 78.5% | 86.2% | 80.4% | 89.0% |

Some of the prior art methods, for example using only “match,” are fairly good at precision (rejecting concepts that aren't present) but not very good at recall (finding all of the concepts that are present). While other prior art methods, for example cTAKES, which uses “match phrase” but not “match” are fairly good at recall, but not so good at precision. Allowing some “slop” with “match phrase” allows the algorithm to find almost as many of the concepts that are present, as using only “match,” while being much better at rejecting concepts that are not present. Using a hybrid algorithm that combines both a match score and a match phrase score, and using two-word queries for the match phrase score and test, so that all words in the concept need not be present, further improves the recall, while using a hybrid algorithm with zero slop in the match phrase test, requiring all words in the concept to be present in the same order, further increases the precision. The full algorithm, which uses queries for the match phrase score and test, and uses mandatory words, has the highest precision and the highest recall, with both being close to 89%. The fact that precision and recall are nearly the same, for the full algorithm, reflects the fact that when the algorithm misses a concept that is present, it almost always finds another concept that is not really present, and vice versa.

It should be noted that, for this test data using the full algorithm, about 27% of the errors (false positives and false negatives) were due to the algorithm finding a concept that is closely related to the concept that was found manually, but either concept is acceptable, and the concept found by the algorithm could reasonably not be considered an error at all. A typical example of this is finding “fracture of radius and/or ulna” instead of finding the two concepts “fracture of radius” and “fracture of ulna”. If these errors were not counted as errors, then F1 would be even higher, about 92%.

Although minor improvements in performance might still be made by fine tuning of the algorithm, the inventor believes that it might not be possible to substantially improve the performance of the algorithm without making use of a deep AI-like understanding of each of the concepts.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of determining which concepts in a set of medical concepts pertain to an input text, automatically by executing instructions on a computer, the method comprising:
a) creating a set of one or more queries for each concept that has two or more words, each query being a string of two words that is a sub-string of the words in the concept, in the same order as the words in the concept;
b) for each concept in a selected sub-set of the concepts that have two or more words, for each query, determining whether or not the input text includes all the words of that query, and if it does, calculating a sub-score indicating a degree of matching between the query and the input text;
c) for each concept in the selected sub-set, for which more than a minimum number of the queries have all of their words in the input text sufficiently close together according to a criterion, calculating a score to indicate a degree of matching between the concept and the input text, depending on the sub-scores of the queries for that concept; and
d) applying one or more rules to determine which of the concepts in the selected sub-set, for which a score was calculated, pertain to the input text and which do not, at least some of the rules depending on the score of the concept.

2. A method according to claim 1, also comprising calculating a match score at least for each concept in the selected sub-set that has two or more words, of which more than a minimum number of the words are in the input text, and for which more than the minimum number of the queries have all of their words in the input text sufficiently close together according to the criterion, the match score indicating a degree of matching between the concept and the input text according to a bag-of-words method, and wherein calculating the score for each concept comprises calculating the score depending on the match score for that concept as well as on the sub-scores of the queries for that concept.

3. A method according to claim 2, wherein calculating the score for each concept comprises calculating a weighted sum of the match score and the sub-scores for the queries.

4. A method according to claim 2, wherein the minimum number of words is 2 for concepts with two words, 2 or 3 for concepts with three words, 2, 3 or 4 for concepts with four words, 3 or 4 for concepts with five words, and 3, 4 or 5 for concepts with six words.

5. A method according to claim 2, also comprising assigning a score to the concepts that have only one word, when the one word is found in the input text, wherein the rules that depend on the score of the concept are applied both to the concepts with only one word and to the concepts with two or more words.

6. A method according to claim 1, wherein the one or more rules specify that when two concepts for which scores have been calculated have sufficiently great overlap in their words, then the concept with a lower score does not pertain to the input text, and that a concept does pertain to the input text if it has a calculated score that is higher than the score calculated for any other concept with which it has sufficiently great overlap in its words.

7. A method according to claim 1, wherein the first minimum number is between 35% and 65% of the number of queries created for that concept.

8. A method according to claim 1, wherein calculating the score comprises calculating a weighted sum of the sub-scores of the queries, with lower weight given to queries with words that are further apart in the concept.

9. A method according to claim 1, wherein the selected sub-set of concepts excludes at least those concepts for which one or more words defined as mandatory words for that concept are not found in the input text.

10. A method according to claim 9, wherein the word in the concept that is rarest among all the words in the set of concepts is defined as a mandatory word.

11. A method according to claim 9, wherein any singular word in the concept that is a name of a disease is defined as a mandatory word.

12. A method according to claim 1, wherein the selected sub-set of concepts excludes at least those concepts which include a word for a body part and a word describing a location or direction of the body part, for which the word that describes the location or direction of a body part is more than one word away in the input text from the word for the body part.

13. A method according to claim 1, wherein the criterion for words in the query being sufficiently close together in the input text specifies a maximum distance between the words that is lower for words that are not in the same order in the input text as they are in the query, and that is higher for words that are spaced further apart in the concept than for words that are spaced closer together in the concept.

14. A method according to claim 13, wherein the maximum distance for words that are adjacent in the concept and are in the same order in the query and in the input text is between 10 and 25.

15. A method according to claim 1, also comprising preparing the set of concepts and preprocessing the input text, comprising:
a) providing an initial set of concepts from a database of medical concepts;
b) modifying the initial set of concepts by expanding vertebrae letter-number designations to include the words “vertebra” and “spine” and replacing the letter of the letter-number designation by the body region that it stands for, cervical, thoracic, or lumbar; and
c) preprocessing the input text by expanding vertebrae letter-number designations to include the words “vertebra” and “spine” and replacing the letter of the letter-number designation by the body region that it stands for, cervical, thoracic, or lumbar.

16. A computer storage product having at least one computer storage medium having instructions stored therein causing one or more computers to perform the method of claim 1.

17. A computer storage medium having instructions stored therein for causing a computer to perform the method of claim 1.

18. A computer product embodied in a computer readable medium for performing the steps of claim 1.

19. A system for automatically identifying which concepts in a set of medical concepts are found in a medical report, the system comprising:
a) a first database that provides access to one or more medical reports;
b) a splitter module with access to the first database that divides a medical report into input texts;
c) a second database that provides access to the set of medical concepts;
d) a processor module, with access to the input texts and to the second database, configured, for each input text, to:
1) create a set of one or more queries for each concept that has two or more words, each query being a string of two words that is a sub-string of the words in the concept, in the same order as the words in the concept;
2) for each concept in a selected sub-set of the concepts that have two or more words, for each query, determine whether or not the input text includes all the words of that query, and if it does, calculate a sub-score indicating a degree of matching between the query and the input text;
3) for each concept in the selected sub-set, for which more than a minimum number of the queries have all of their words in the input text sufficiently close together according to a criterion, calculate a score to indicate a degree of matching between the concept and the input text, depending on the sub-scores of the queries for that concept; and
4) apply one or more rules to determine which of the concepts in the selected sub-set, for which a score was calculated, pertain to the input text and which do not, at least some of the rules depending on the score of the concept; and
e) an output module that outputs the concepts that are determined to pertain to the input texts of the medical report.

20. A method of determining which concepts in a set of medical concepts pertain to an input text, automatically by executing instructions on a computer, the method comprising:
a) for each concept in the set, applying one or more criteria for determining whether of not the concept is a possible match for the input text, wherein, for at least some of the concepts, the criteria do not require that all of the words of the concept are found in the input text, but do require that one or more words specified to be mandatory words of the concept are found in the input text; and
b) applying one or more rules to determine which of the concepts that are possible matches to the input text pertain to the input text, and which do not.

21. A method according to claim 20, also including calculating a score, for each concept that is a possible match to the input text, indicating a degree of matching between the concept and the input text, wherein at least some of the one or more rules depend on the score of the concept and the scores of any other concepts that are possible matches to the input text.

22. A method according to claim 20, wherein a word of the concept that is least common among all the concepts in the set is specified to be a mandatory word for that concept.

23. A method according to claim 20, wherein a word of a concept that is a one-word term for a disease is specified to be a mandatory word for that concept.

24. A method according to claim 20, wherein, for a concept that includes a word for a body part and a word that describes a location of direction of the body part, both words are specified to be mandatory words for that concept, and the criteria further require that the words are adjacent to each other in the input text.

25. A method of determining which concepts in a set of medical concepts pertain to an input text, automatically by executing instructions on a computer, the method comprising:
a) for each concept in the set, applying criteria for determining whether or not the concept is a possible match for the input text;
b) for each concept that is a possible match to the input text, calculating a score indicating a degree of matching between the concept and the input text; and
c) determining that, when two concepts for which scores have been calculated have sufficiently great overlap in their words, then the concept with a lower score does not pertain to the input text, and that a concept does pertain to the input text if it has a calculated score that is higher than the score calculated for any other concept with which it has sufficiently great overlap in its words;
wherein, for at least some of the concepts in the set, calculating the score comprises calculating a match score, calculating a match phrase score, and combining the match score and the match phrase score to obtain the score, the match score depending on how many of the words in the concept are found in the input text but not on the order of those words in the input text, and the match phrase score depending both on how many of the words in the concept are found in the input text, and on the order of those words in the input text.

26. (canceled)

27. A system for automatically identifying which concepts in a set of medical concepts are found in a medical report, the system comprising:
a) a first database that provides access to one or more medical reports;
b) a splitter module with access to the first database that divides a medical report into input texts;
c) a second database that provides access to the set of medical concepts;
d) a processor module, with access to the input texts and to the second database, configured, for each input text, to:
1) for each concept in the set, applying one or more criteria for determining whether of not the concept is a possible match for the input text, wherein, for at least some of the concepts, the criteria do not require that all of the words of the concept are found in the input text, but do require that one or more words specified to be mandatory words of the concept are found in the input text; and
2) applying one or more rules to determine which of the concepts that are possible matches to the input text pertain to the input text, and which do not; and
e) an output module that outputs the concepts that are determined to pertain to the input texts of the medical report.

28. A system for automatically identifying which concepts in a set of medical concepts are found in a medical report, the system comprising:
a) a first database that provides access to one or more medical reports;
b) a splitter module with access to the first database that divides a medical report into input texts;
c) a second database that provides access to the set of medical concepts;
d) a processor module, with access to the input texts and to the second database, configured, for each input text, to:
1) for each concept in the set, applying criteria for determining whether or not the concept is a possible match for the input text;
2) for each concept that is a possible match to the input text, calculating a score indicating a degree of matching between the concept and the input text; and
3) determining that, when two concepts for which scores have been calculated have sufficiently great overlap in their words, then the concept with a lower score does not pertain to the input text, and that a concept does pertain to the input text if it has a calculated score that is higher than the score calculated for any other concept with which it has sufficiently great overlap in its words;
wherein, for at least some of the concepts in the set, calculating the score comprises calculating a match score, calculating a match phrase score, and combining the match score and the match phrase score to obtain the score, the match score depending on how many of the words in the concept are found in the input text but not on the order of those words in the input text, and the match phrase score depending both on how many of the words in the concept are found in the input text, and on the order of those words in the input text, and
e) an output module that outputs the concepts that are determined to pertain to the input texts of the medical report.
